(19) United States
(12) Patent Application Publication
(10) Pub. No.: US 2016/0166603 A1
(43) Pub. Date: Jun. 16, 2016

(54) AGENT AND METHOD FOR TREATING HERPES

(71) Applicant: NAJÖPHARM GMBH, Hamburg (DE)

(72) Inventor: Nadine Heiser, Hamburg (DE)

(21) Appl. No.: 14/891,102

(22) PCT Filed: May 14, 2014
(86) PCT No.: PCT/EP2014/059813

§ 371 Date: Nov. 13, 2015

(30) Foreign Application Priority Data
May 14, 2013 (EP) 13167597.7

(51) Int. Cl. A61K 31/78; A61K 9/00; A61K 31/133

(52) U.S. Cl. CPC A61K 31/78; A61K 31/133; A61K 9/0014

(57) ABSTRACT

The invention relates to a therapeutic active substance combination comprising polyacrylic acid and 2-amino-2-methylpropanol for use as a drug, especially for the prophylactic and/or therapeutic treatment of herpes infections.

The present invention relates to a composition and a therapeutic agent combination with effect against viruses of the herpes family, and a method for using this composition or therapeutic agent combination for treatment in the prophylactic and/or therapeutic treatment of herpes infections.

### BACKGROUND OF THE INVENTION

The herpes virus family includes the herpes simplex virus type HSV-1 and HSV-2 and the varicella-zoster virus (VZV). The primary infection with HSV typically causes lesions at the site of infection, which usually heal within 2-3 weeks, after which the virus travels to the ganglia, where it remains in a latent phase until activation. The activation, triggered by a stimulus such as stress, cold, fever, immune deficiency, excessive sun exposure, poor nutrition, menstruation, etc. leads to secondary outbreaks (recurrent infection), which typically manifests itself with HSV-1 on the lips as cold sores and typically causes lesions on the genitals or in the surrounding region with HSV-2. Infections with HSV serotypes are prevalent. Thus, an estimated 90% of people carry HSV-1 and many of the virus carriers will experience the painful fever blister on the lips at least once in their lives [W. Hellenbrand et al.: Seroprevalence of herpes simplex virus type 1 (HSV-1) and type 2 (HSV-2) in former East and West Germany, 1997-1998; Eur. J. Clin. Microbiol. Infect. Dis. (2005) 24(2): p. 131-135].

According to the current state of knowledge, healing the aforementioned herpes infections is not possible. Antiviral therapies reduce the symptoms of secondary outbreaks in patients to an unsatisfactory degree. Particularly affected by this are “untreatable” patients, in whom acyclovir resistant herpes strains have been isolated in the course of treatment with, for example, the currently most commonly used antivirals acyclovir [2-amino-9-(2-hydroxyethoxy)methyl-1,9-dihydro-6H-purin-6-on, commercially available under Zovirax® and the like].

There is therefore a need for new substances and methods for the treatment of herpes infections.

### DESCRIPTION OF THE INVENTION

The object of the present invention is to provide an effective agent as an alternative or supplement to the treatment options for herpes infection known from the prior art.

This object is achieved by a composition according to the claims and therapeutic agent combination comprising polyacrylic acid and 2-amino-2-methylpropanol, for use as medication, particularly for the prophylactic and/or therapeutic treatment of herpes infections in accordance with the independent claims. Advantageous embodiments of the invention are disclosed in the dependent claims. Surprisingly, it has been shown that the inventive composition or therapeutic agent combination can reduce the duration of a herpes infection, and is also suitable for the prophylactic suppression of recurrent herpes.

First, some terms used in the present invention will be explained.

The inventive composition or therapeutic agent combination is used to treat a subject or individual who suffers from a herpes infection, in particular herpes labialis (cold sores), gingivostomatitis herpetica (mouth ulcers) or herpes simplex genitalis (genital herpes). The treated herpes infection can be caused by the herpes simplex virus type 1 or 2 or varicella zoster virus (herpes zoster). The term “subject” includes live animals and humans. The purpose of this treatment is to at least partially alleviate the symptoms of herpes infection and in particular the to shorten the recurrent phases, as summarized briefly below for cold sores (herpes labialis): In the prodromal phase (day 0-1), the following symptoms typically occur: pain, tingling, burning, tightness and itching that are correlated in time with the early phase of viral replication in the skin, which takes place mainly in the first 48 hours. In the ensuing papule phase (day 2-3), the skin initially reddens before more or less thick papules form on the edge of the lips that are reddened and painful for the patient, which develop in the vesicular phase into liquid-filled blisters with a diameter of between 2-5 mm and then break open and merge to create painfully weeping ulcers in the ulceration phase (day 4). Towards the end of the infection, there then follows the incrustation (day 5-8) and healing phase (day 9-14), during which new skin forms beneath the crust. Untreated, the cold sore is usually fully healed after 2 weeks. A dreaded variant of the cold sore is an involvement of the eye, particularly the cornea, as a result of which corneal opacities and corneal scarring can occur, which are associated with impaired vision and even blindness.

The present invention also relates to the treatment of a subject suffering from a herpes infection, wherein a therapeutically effective amount of the inventive composition or combination of agents is administered to the subject.

The term “treatment” in the context of the present invention refers to the full or partial achievement of the following results: whole or partial reduction of the clinical picture; improvement of at least one of the clinical symptoms or the indicators associated with the disease; delay, suppression or protection against the progression of the disease; or total or partial delay, suppression or protection against the outbreak or development of the disease. The subject to be treated is a human or animal, preferably a mammal.

The term “composition comprising polyacrylic acid and 2-amino-2-methylpropanol” includes the composition as a combination product of the two aforementioned therapeutically active components without further therapeutically active components. In addition to the therapeutically active agents polyacrylic acid and 2-amino-2-methylpropanol, however, the composition may also comprise at least one additional therapeutically active substance. The composition of the invention can be administered alone or in combination with at least one other therapeutic substance to reduce one or more symptoms of the herpes infection. The administration of the composition according to the invention can simultaneously take place with the other therapeutic substance (e.g. essential oils, such as of lemon balm, peppermint; cutleaf self-heal, rosemary, sage or thyme, tea tree and eucalyptus), which may be a component of the same composition or provided in another composition. Alternatively, the composition of the invention may be administered before or after administration of the other therapeutic substance. The inventive composition may be administered via the same or a different administration route as the other therapeutic substance.

The inventive composition is preferably provided and administered as a pharmaceutical composition. The term “pharmaceutical composition” comprises the active ingredient combination of polyacrylic acid and 2-amino-2-methylpropanol and one or more inert ingredients which act as carriers for the active ingredients.

Polyacrylic acid and 2-amino-2-methylpropanol are known from the prior art, for example WO-A-2011/015 628 and US-A-2013/0072575, but not as a therapeutically active agent combination in the sense of the present invention.

The pharmaceutical composition is preferably formulated so that it can be applied in the context of local therapy or topical therapy. The agents therefore act only where they are needed, while the infection-free areas are spared. The pharmaceutical composition may be applied directly to the affected areas of the herpes infection. Alternatively, or in addition, the pharmaceutical composition may also be administered via a depot placed on the affected areas, for example, a depot plaster coated or impregnated with the pharmaceutical composition.

A form of administration may be, for example, a tincture, aerosol, solution, cream, paste, lotion, gel or ointment. Appropriate techniques for formulation and administration are known from the prior art, see for example “Remington's Pharmaceutical Sciences” (Mack Publishing Co., Easton Pa.). In general, the agent is applied by the patients 1 to 20 times daily, preferably more than 3 times daily, particularly preferably 5 to 15 times daily to the affected areas. Particularly preferred is a thin film application. A drying of the film is recommended. For prophylactic treatment, at least one weekly application on former sites of infection is recommended. In the course of treatment, the infection is prevented in the prodromal stage, while when used in the erythema stage, the goal is to reduce symptoms and prevent the formation of blisters; with already existing papules, vesicles or ulcers, the application of the composition of the invention leads to the resolution of infection and the rapid healing of skin lesions.

The proportion of the active ingredients polyacrylic acid and 2-amino-2-methylpropanol in the pharmaceutical composition can vary, and is usually based on the total weight of the composition at 0.05 to 15 wt.-%, preferably 0.1-5 wt.-%, more preferably 0.5-2.5 wt.-%, more preferably 0.75 to 1.25 wt.-%, more preferably 1 wt.-% polyacrylic acid and 0.05-15 wt.-%, preferably 0.1-5 wt.-%, more preferably 0.5-2.5 wt.-%, more preferably 0.75 to 1.25 wt.-%, particularly preferably 1 wt.-% of 2-amino-2-methylpropanol. The active ingredient content is accordingly selected so that an effective dose is reached.

The polyacrylic acid and 2-amino-2-methylpropanol can be used in “pure form” or “purified,” which means that unwanted components, such as the toxic solvent benzene used in the preparation of polyacrylic acid, have been removed.

“Polyacrylic acid” (CAS number 9007-20-9, Merck Index 11, December 1989, ISBN 0-911910-28-X) has the basic structure reproduced below

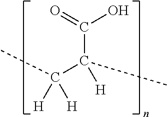

and is a synthetic compound and high molecular weight polymer of acrylic acid occurring in various degrees of polymerization (molecular weight), which are all suited for the purposes of the present invention. Examples include its benzene-free commercial types Carbopol 981, Carbopol 971, Carbopol 71G, Carbopol 974P, Carbopol 984, Carbopol 5984, Carbopol 980, Carbopol 934P, Carbopol 941, Carbopol 971, Carbopol 940 or Carbopol ETD 2001, 2020 and 2050. The above commercial goods corresponds to the pharmaceutical pharmacopoeias, see Ph. Eur. 01/2005:1299. Polyacrylic acid is well tolerated by the skin and also applicable to mucous membranes. Polyacrylic acid is synthesized as polymerization initiators by free radical polymerization in the presence of peroxides with azo compounds or other radical forming substances. The preparation of polyacrylic acid is known to the person skilled in the art and described, for example, in Bracher et al., Arzneibuch-Kommentar [Pharmacopeia commentary] (2010), ISBN 3-8047-2115-X.

2-amino-2-methylpropanol (CAS number 124-68-5) has the structure reproduced below:

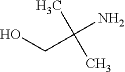

2-amino-2-methylpropanol may be obtained by reduction of the corresponding nitro compound, as described for example in U.S. Pat. No. 2,174,242.

The above-described compositions of the invention comprise the two therapeutic agents polyacrylic acid and 2-amino-2-methylpropanol as components of one composition. However, this is not absolutely necessary, that is, both therapeutic agents may be present spatially separated from one another in differing compositions (i.e. at least 2) and still exert their therapeutic effect in combination of both therapeutic agents, for example, in simultaneous or sequential treatment of the herpes infection with the compositions each comprising one of the two components (polyacrylic acid on the one hand and 2-amino-2-methylpropanol on the other) of the therapeutic agent combination. The therapeutic agent polyacrylic acid may be a component of a first composition and the therapeutic agent 2-amino-2-methylpropanol a component of a second composition, wherein both compositions are present as a collection spatially separated from one another as a so-called “kit-of-parts” and exert their therapeutic effect in the application of both compositions. Both compositions of the kit-of-parts may be used simultaneously (separately or as a previously prepared mixture of both compositions) or sequentially. A sequential administration includes a directly successive application of the compositions.

According to a preferred embodiment of the invention, the pharmaceutically active agent combination of polyacrylic acid and 2-amino-2-methylpropanol is used in a galenic preparation which may contain one or more pharmaceutical adjuvants and/or additives, preferably selected from the group consisting of a pharmaceutically acceptable carrier, fillers, fragrances and stabilizers. The pharmaceutical formulation of the medicament preferably allows a controlled and/or delayed release of the pharmaceutically active ingredients. According to a preferred embodiment of the invention, in addition to the pharmaceutically active agent combination of polyacrylic acid and 2-amino-2-methylpropanol, the composition comprises the galenic adjuvants ethanol, glycerol and water, preferably distilled water. Based on the total weight of the composition, this may contain
20-70 wt.-%, preferably 30-60 wt.-%, more preferably 40-50 wt.-%, particularly preferably 45 wt.-% of ethanol,
5-45 wt.-%, preferably 15-35 wt.-%, more preferably 20-30 wt.-%, particularly preferably 25 wt.-% of glycerol and
5-45 wt.-%, preferably 15-35 wt.-%, more preferably 20-30 wt.-%, particularly preferably 25 wt.-% of water.

A number of exemplary combinations of the amounts of the individual components, as may be included in the composition of the invention, are given in tabular form below. It is obvious to the person skilled in the art that the individual components can add up to a maximum of 100, i.e. the upper ranges of all of the components included in the pharmaceutical formulation (ethanol, glycerol and water) cannot be used in practice at the same time.

| | Examples [figures in wt.-% based on the total | | | | |
|---|---|---|---|---|---|
| | weight of the composition] | | | | |
| Components | 1 | 2 | 3 | 4 | 5 |
| Polyacrylic | 0.05-15 | 0.1-10 | 0.1-5 | 0.5-2.5 | 0.75-1.25 |
| acid | | | | | |
| 2-amino-2- | 0.05-15 | 0.1-10 | 0.1-5 | 0.5-2.5 | 0.75-1.25 |
| methylpropanol | | | | | |
| Ethanol | 20-70 | 30-60 | 30-60 | 40-50 | 40-50 |
| glycerol | 5-45 | 15-35 | 15-35 | 20-30 | 20-30 |
| Water | 5-45 | 15-35 | 15-35 | 20-30 | 20-30 |
| Components | 6 | 7 | B | 9 | 10 |
| Polyacrylic | 0.05-15 | 0.1-10 | 0.2-5 | 0.5-2.5 | 0.75-1.25 |
| acid | | | | | |
| 2-amino-2- | 0.05-15 | 0.1-10 | 0.2-5 | 0.5-2.5 | 0.75-1.25 |
| methylpropanol | | | | | |
| Ethanol | 30-60 | 30-60 | 30-60 | 30-60 | 30-60 |
| glycerol | 15-35 | 15-35 | 15-35 | 15-35 | 15-35 |
| Water | 15-35 | 15-35 | 15-35 | 15-35 | 15-35 |
| Components | 11 | 12 | 13 | 14 | 15 |
| Polyacrylic | 0.05-15 | 0.1-10 | 0.2-5 | 0.5-2.5 | 0.75-1.25 |
| acid | | | | | |
| 2-amino-2- | 0.05-15 | 0.1-10 | 0.2-5 | 0.5-2.5 | 0.75-1.25 |
| methylpropanol | | | | | |
| Ethanol | 40-50 | 40-50 | 40-50 | 40-50 | 40-50 |
| glycerol | 15-35 | 15-35 | 15-35 | 15-35 | 15-35 |
| Water | 15-35 | 15-35 | 15-35 | 15-35 | 15-35 |
| Components | 16 | 17 | 18 | 19 | 20 |
| Polyacrylic | 0.05-15 | 0.1-10 | 0.2-5 | 0.5-2.5 | 0.75-1.25 |
| acid | | | | | |
| 2-amino-2- | 0.05-15 | 0.1-10 | 0.2-5 | 0.5-2.5 | 0.75-1.25 |
| methylpropanol | | | | | |
| Ethanol | 30-60 | 30-60 | 30-60 | 30-60 | 30-60 |
| glycerol | 20-30 | 20-30 | 20-30 | 20-30 | 20-30 |
| Water | 20-30 | 20-30 | 20-30 | 20-30 | 20-30 |
| Components | 21 | 22 | 23 | 24 | 25 |
| Polyacrylic | 0.05-15 | 0.1-10 | 0.2-5 | 0.5-2.5 | 0.75-1.25 |
| acid | | | | | |
| 2-amino-2- | 0.05-15 | 0.1-10 | 0.2-5 | 0.5-2.5 | 0.75-1.25 |
| methylpropanol | | | | | |
| Ethanol | 40-50 | 40-50 | 40-50 | 40-50 | 40-50 |
| glycerol | 20-30 | 20-30 | 20-30 | 20-30 | 20-30 |
| Water | 20-30 | 20-30 | 20-30 | 20-30 | 20-30 |

The ethanol component of the composition according to the invention may be at least partially denatured with a denaturing agent, wherein the denaturant is preferably selected from the group consisting of propanone, butanone, denatonium benzoate, mint oil, petroleum ether, toluene, cyclohexane, diethyl phthalate and pyridine.

The composition of the invention may further comprise penetration-enhancing adjuvants, such as propylene glycol or dimethyl sulfoxide.

It is particularly preferred in the context of the invention when the pH of the final composition for the application in aqueous solution is 7-9, preferably 7.5-8.5, more preferably 7.75 to 8.25, and particularly preferably 8 (measurement according to DIN ISO 10390:2005-12). The preparation of the composition of the invention is effected under sterile conditions. Well-known and established methods (mixing, stirring, homogenizing and filtering) are used in this context as well as standard equipment (see for example Remington's Pharmaceutical Sciences and U.S. Pharmacopeia and Viogt, Rudolf, Pharmazeutische Technologie für Studium and Beruf [Pharmaceutical technology for study and work], 7th Edition, Berlin (1993). In the course of the preparation, the mixing of the ingredients may preferably take place by stirring. Preferred periods for mixing are 1 to 20 h.

The inventive composition may be packaged as a single or multiple dose. The best dosage for each patient is determined by, for example, the general condition of the patient, disease progression and the form of application. In the case of individuals whose herpes recur very frequently, usually more than six times per year, and who are therefore seriously affected in their well-being, a prophylactic long-term suppression therapy with the inventive composition may be considered, which is preferably provided to the patient in a multi-dose packaging for self-medication.

The invention will now be further described in the examples below, without being limited thereto.

### EXAMPLES

### Example 1

The inventive formulation below with a slightly alkaline pH of 7.8 was prepared under cleanroom conditions at a two-hour mixing of the components at 20′ C. The gel-like solution is filled into sterile containers to be subsequently applied to the skin of patients using a brush, spatula or fingers (see Example 2).

| | |
|---|---|
| Polyacrylic acid (CAS No. 9007-20-9) 2- | 1 wt.-% |
| amino-2-methylpropanol (CAS No. 124-68-5) | 1 wt.-% |
| denat. alcohol (CAS No. 64-17-5) | approx. 45 wt.-% |
| glycerol (CAS No. 56-81-5) | approx. 25 wt.-% |
| Dist. water (CAS No. 7732-18-5) | approx. 25 wt.-% |

### Example 2

| | |
|---|---|
| Case 1: | |
| ♀, 31 years old, | |
| Diagnosis: | chronic herpes labialis |
| | occasional herpes genitalis |

1st visit Mar. 15, 2013

Typical lip infection in the papule phase
Typical left lateral infection of labia minora
Instructions for treatment

2nd visit Mar. 18, 2013

Lip largely now only slightly reddened Genital
infection healing extensively

The following application notes are based on medical case studies (cases 1-3) and own experiences of patients where the inventive composition according to example 1 was provided for self-medication (cases 4-8).

3rd visit Mar. 22, 2013
No further symptoms detectable

Case 2:
♂, 21 years old,
Diagnosis: herpes labialis after the 4th outbreak in the first
year
1. 1st visit Mar. 22, 2013

Erythema phase
Instructions for treatment

2. 2nd visit Mar. 25, 2013

| | No symptoms |
|---|---|
| Case 3: | |

♀, 51 years old,
Diagnosis: condition after TB treatment 2 years ago

Now approx. 6th outbreak of herpes labialis

1. 1st visit Apr. 3, 2013

Prodromal stage lip exhibiting tingling in previous
skin area at bottom right
Instructions for treatment

2. 2nd visit Apr. 6, 2013

| | No further symptoms detectable |
|---|---|
| Case 4: | |
| Subject | |
| Gender: | male |
| Age: | 42 |
| Disease | |
| Designation: | herpes labialis (cold sores) |
| Stage/symptoms: | initial blistering; |
| | itching/inflammatory pain |
| Application | |
| Form: | topical application directly to the |
| | affected skin area |
| Frequency: | at least 10 times a day |

Progress of the cure
No further blister growth; reduction of
itching/inflammatory pain; blisters dried out within a few
hours; all symptoms disappeared after three days

| | |
|---|---|
| Case 5: | |
| Subject | |
| Gender: | male |
| Age: | 42 |
| Disease | |
| Designation: | herpes labialis (cold sores) |
| Stage/symptoms: | initial reddening and localized |
| | itching/inflammatory pain; no blistering |
| | yet |
| Application | |
| Form: | topical application directly to |
| | the affected skin area |
| Frequency: | at least 10 times a day |

Progress of the cure
No blistering; reduction of itching/
inflammatory pain; all symptoms disappeared after one
day

| | |
|---|---|
| Case 6: | |
| Subject | |
| Gender: | female |
| Age: | 39 |
| Disease | |
| Designation: | herpes labialis (cold sores) |
| Stage/symptoms: | initial blistering; itching/inflammatory |
| | pain |
| Application | |
| Form: | topical application directly to the |
| | affected skin area |
| Frequency: | at least 10 times a day |

Progress of the cure
No further blister growth; reduction of itching/inflammatory
pain; blisters dried out within a few hours; all symptoms
disappeared after three/four days

| | |
|---|---|
| Case 7: | |
| Subject | |
| Gender: | female |
| Age: | 40 |
| Disease | |
| Designation: | herpes labialis (cold sores) |
| Stage/symptoms: | advanced blistering; |
| | itching/inflammatory pain |
| Application | |
| Form: | topical application directly to the |
| | affected skin area |
| Frequency: | at least 10 times a day |

Progress of the cure
No further blister growth; reduction of
itching/inflammatory pain; blisters dried out within a few
hours; all symptoms disappeared after three days

| | |
|---|---|
| Case 8: | |
| Subject | |
| Gender: | female |
| Age: | 32 |
| Disease | |
| Designation: | herpes labialis (cold sores) |
| Stage/symptoms: | initial blistering; |
| | itching/inflammatory pain |
| Application | |
| Form: | topical application directly to the |
| | affected skin area |
| Frequency: | at least 10 times a day |

Progress of the cure
No further blister growth; reduction of itching/inflammatory
pain; blisters dried out within a few hours; all symptoms
disappeared after three/four days

It has been found that a significant reduction in the duration of the outbreaks could be achieved by administration of the inventive composition according to Example 1. The duration of the vesicular stage was shortened, the lesions healed faster and the pain, itching and burning were favorably influenced. No contraindications or side effects occurred. Comparing the results with early initiation of therapy (prodromal or erythema stage) with those with late initiation of therapy (papule and vesicle stage), it can be concluded that the healing was accelerated regardless of the stage at the time of initiation of therapy. Even in the case of late applications in the papule and vesicle phase, the composition of the invention showed a significant effect on the healing time of lesions. Thus, the examples show a therapeutic effect that is largely independent from the start of treatment.

1. A composition comprising
a) polyacrylic acid and
b) 2-Amino-2-methylpropanol
as therapeutic agents for use as medication.

2. A composition comprising
a) polyacrylic acid and
b) 2-amino-2-methylpropanol
as therapeutic agents for use in the prophylactic and/or therapeutic treatment of herpes infections.

3. A composition for use according to claim 2, characterized in that the herpes infection to be treated is herpes simplex labialis (cold sores), gingivostomatitis herpetica (mouth ulcers) or herpes simplex genitalis (genital herpes).

4. A composition for use according to claim 2, characterized in that the herpes infection to be treated is an infection with a virus that is selected from the group consisting of
herpes simplex virus type 1,
herpes simplex virus type 2 and
varicella-zoster virus (herpes zoster).

5. A composition for use according to any one of the preceding claims, characterized in that the composition contains, based on the total weight of the composition:
a) 0.05-15 wt.-%, preferably 0.1-5 wt.-%, more preferably 0.5-2.5 wt.-%, more preferably 0.75-1.25 wt.-%, particularly preferably 1 wt.-% polyacrylic acid and
b) 0.05-15 wt.-%, preferably 0.1-5 wt.-%, more preferably 0.5-2.5 wt.-%, more preferably 0.75-1.25 wt.-%, particularly preferably 1 wt.-% 2-amino-2-methylpropanol.

6. A composition for use according to any one of the preceding claims, further comprising one or more pharmaceutical adjuvants and/or additives, preferably selected from the group consisting of a pharmaceutically acceptable carrier, fillers, fragrances and stabilizers.

7. A composition for use according to any one of the preceding claims comprising
c) ethanol,
d) glycerol and
e) water.

8. A composition for use according to claim 7, characterized in that the composition contains, based on the total weight of the composition:
c) 20-70 wt.-%, preferably 30-60 wt.-%, more preferably 40-50 wt.-%, particularly preferably 45 wt.-% ethanol,
d) 5-45 wt.-%, preferably 15-35 wt.-%, more preferably 20-30 wt.-%, particularly preferably 25 wt.-% glycerol and
e) 5-45 wt.-%, preferably 15-35 wt.-%, more preferably 20-30 wt.-%, particularly preferably 25 wt.-% water.

9. A composition for use according to claim 7 or 8, characterized in that component c) is at least partially denatured with a denaturing agent, wherein the denaturant is preferably selected from the group consisting of propanone, butanone, denatonium benzoate, mint oil, petroleum ether, toluene, cyclohexane, diethyl phthalate, and pyridine.

10. A composition for use according to any one of the preceding claims, characterized in that the composition is formulated for topical administration.

11. A composition for use according to any one of the preceding claims, characterized in that the administration form is selected from the group consisting of tincture, gel, paste, lotion, ointment, cream or aerosol.

12. A composition for use according to any one of the preceding claims, characterized in that the composition in aqueous solution has a pH of 7-9, preferably 7.5-8.5, more preferably 7.75 to 8.25, particularly preferably 8.

13. A composition for use according to any one of the preceding claims, characterized in that the composition is packaged as a single or multiple dose.

14. A therapeutic agent combination comprising
a) polyacrylic acid and
b) 2-amino-2-methylpropanol
for use as medication, wherein said polyacrylic acid is a component of a first composition and said 2-amino-2-methylpropanol is a component of a second composition and the first and the second composition are present separated from each other spatially as a collection (kit-of-parts).

15. A therapeutic agent combination comprising
a) polyacrylic acid and
b) 2-amino-2-methylpropanol
for use in the prophylactic and/or therapeutic treatment of herpes infections,
wherein said polyacrylic acid is a component of a first composition and said 2-amino-2-methylpropanol is a component of a second composition and the first and the second composition are present separated from each other spatially as a collection (kit-of-parts).
